SEQUENCE LISTING (6 sequences)

SEQ ID NO: 1 - DNA, 5724 bp, Homo sapiens
ggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaaaagatttaaacaggatggtggctggagccactggtccccgtggtcatcttgttctgtgacatgtggtgatggtgtgatcacaaggatccggctctgcaactctcccagcccccagatgaatgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccgcaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgttttttttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatattt

SEQ ID NO: 2 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPAPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 3 - DNA, 5784 bp, Homo sapiens
acggcatccagtacagaggggctggacttggacccctgcagcagccctgcacaggagaagcggcatataaagccgcgctgcccgggagccgctcggccacgtccaccggagcatcctgcactgcagggccggtctctcgctccagcagagcctgcgcctttctgactcggtccggaacactgaaaccagtcatcactgcatctttttggcaaaccaggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcataggaccagttgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctcgatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggcttctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggtcacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgctggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagaccggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctagacacctcagttcattgtggtccttgcggcttctctctctagcagcacctcctgtcccttgaccttaactctgatggttcttcacctcctgccagcaaccccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctctacaatgacacacaaatcccgctaaataaattataaacaagggtcaattcaaatttgaagtaatgttttagtaaggagagattagaagacaacaggcatagcaaatgacataagctaccgattaactaatcggaacatgtaaaacagttacaaaaataaacgaactctcctcttgtcctacaatgaaagccctcatgtgcagtagagatgcagtttcatcaaagaacaaacatccttgcaaatgggtgtgacgcggttccagatgtggatttggcaaaacctcatttaagtaaaaggttagcagagcaaagtgcggtgctttagctgctgcttgtgccgttgtggcgtcggggaggctcctgcctgagcttccttccccagctttgctgcctgagaggaaccagagcagacgcacaggccggaaaaggcgcatctaacgcgtatctaggctttggtaactgcggacaagttgcttttacctgatttgatgatacatttcattaaggttccagttataaatattttgttaatatttattaagtgactatagaatgcaactccatttaccagtaacttattttaaatatgcctagtaacacatatgtagtataatttctagaaacaaacatctaataagtatataatcctgtgaaaatatgaggcttgataatattaggttgtcacgatgaagcatgctagaagctgtaacagaatacatagagaataatgaggagtttatgatggaaccttaatatataatgttgccagcgattttagttcaatatttgttactgttatctatctgctgtatatggaattcttttaattcaaacgctgaaaacgaatcagcatttagtcttgccaggcacacccaataatcagtcatgtgtaatatgcacaagtttgtttttgtttttgttttttttgttggttggtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacgatccataactttagtcttaatgtacacattgcattttgataaaattaattttgttgtttcctttgaggttgatcgttgtgttgttttgctgcactttttacttttttgcgtgtggagctgtattcccgagacaacgaagcgttgggatacttcattaaatgtagcgactgtcaacagcgtgcaggttttctgtttctgtgttgtggggtcaaccgtacaatggtgtgggaatgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagattaacaacaggaaataaattgtaaaaaaggttttct

SEQ ID NO: 4 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIGPVALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGFCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 5 - DNA, 3074 bp, Homo sapiens
gaattccggggagcaggaagagccaacatgctggccccgcgcggagccgccgtcctcctgctgcacctggtcctgcagcggtggctagcggcaggcgcccaggccaccccccaggtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaagcaaagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggacttcttggaagagctgaagctggtggtgagaggctcactgttccaggtggccagcctgcaagactgcttcctgcagcagagtgagccactggctgccacaggcacaggggactttaaccggcagttcttgggtcaaatgacacaattaaaccaactcctgggagaggtgaaggaccttctgagacagcaggttaaggaaacatcatttttgcgaaacaccatagctgaatgccaggcttgcggtcctctcaagtttcagtctccgaccccaagcacggtggtcgccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtctgcactgacattgatgagtgtcgaaatggagcgtgcgttcccaactcgatctgcgttaatactttgggatcttaccgctgtgggccttgtaagccggggtatactggtgatcagataaggggatgcaaagtggaaagaaactgcagaaacccagagctgaacccttgcagtgtgaatgcccagtgcattgaagagaggcagggggatgtgacatgtgtgtgtggagtcggttgggctggagatggctatatctgtggaaaggatgtggacatcgacagttaccccgacgaagaactgccatgctctgccaggaactgtaaaaaggacaactgcaaatatgtgccaaattctggccaagaagatgcagacagagatggcattggcgacgcttgtgacgaggatgctgacggagatgggatcctgaatgagcaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggaataaaaaacattctggacaactgcccaaaatttcccaatcgtgaccaacgggacaaggatggtgatggtgtgggggatgcctgtgacagttgtcctgatgtcagcaaccctaaccagtctgatgtggataatgatctggttggggactcctgtgacaccaatcaggacagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagcgacggagtgggagacatctgtgagtctgactttgaccaggaccaggtcatcgatcggatcgacgtctgcccagagaacgcagaggtcaccctgaccgacttcagggcttaccagaccgtgggcctggatcctgaaggggatgcccagatcgatcccaactgggtggtcctgaaccagggcatggagattgtacagaccatgaacagtgatcctggcctggcagtggggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatgacaccatccctgaggacttccaagagtttcaaacccagaatttcgaccgcttcgataattaaaccaaggaagcaatctgtaactgcttttcggaacactaaaaccatatatattttaacttcaattttctttagcttttaccaacccaaatatatcaaaacgttttatgtgaatgtggcaataaaggagaagagatcatttttaaaaaaaaaaaaaaaaaaaa

SEQ ID NO: 6 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLSKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVAPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKVERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVGLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

(19) United States
(12) Patent Application Publication
(10) Pub. No.: US 2003/0166017 A1
(43) Pub. Date: Sep. 4, 2003

(54) Compositions and methods for the identification, assessment, prevention and therapy of cardiovascular disease

(75) Inventor: Jeanette McCarthy, San Diego, CA (US)

Correspondence Address: LAHIVE & COCKFIELD, 28 STATE STREET, BOSTON, MA, 02109, US

(73) Assignee: Millennium Pharmaceuticals, Inc., Cambridge, MA

(21) Appl. No.: 10/008,093

(22) Filed: Nov. 9, 2001

Related U.S. Application Data
(60) Provisional application No. 60/248,185, filed on Nov. 9, 2000.
(60) Provisional application No. 60/257,417, filed on Dec. 22, 2000.

(51) Int. Cl. G01N 33/567; C12Q 1/68

(52) U.S. Cl. 435/7.21; 435/6

(57) ABSTRACT

The invention relates to compositions, kits, and methods for detecting, characterizing, preventing, and treating cardiovascular diseases.

### RELATED APPLICATIONS

The present application claims priority to U.S. provisional patent application serial No. 60/248,185, filed on Nov. 9, 2000, and U.S. provisional patent application serial No. 60/257,417, filed on Dec. 22, 2000, both of which are expressly incorporated by reference.

### FIELD OF THE INVENTION

The field of the invention is cardiovascular disease, including diagnosis, characterization, management, and therapy of cardiovascular disease.

### BACKGROUND OF THE INVENTION

Cardiovascular disease (CVD) is the general term for heart and blood vessel diseases including atherosclerosis, coronary heart disease, myocardial infarction (MI), coronary artery disease, stroke, peripheral vascular diseases and congestive heart failure. Cardiovascular disease is a major health risk throughout the industrialized world, accounting for one in every two deaths in developed countries. Almost 60 million Americans suffer from one or more types of cardiovascular diseases. In 1997, CVD caused about 41 percent of all deaths in the United States; about one sixth of all people killed by CVD are under age 65. Atherosclerosis, the most prevalent of cardiovascular diseases, is the principal cause of heart attack, stroke, and gangrene of the extremities, and thereby the principle cause of death in the United States.

The thrombospondins are a family of extracellular adhesive proteins. Five members of this family have been identified: Thrombospondin-1 through 4 and cartilage oligomeric matrix protein (COMP). The thrombospodins play a role in platelet aggregation and adhesion, angiogenesis and other cellular processes. Thrombospondins (also known as thrombin sensitive proteins or TSPs) are large molecular weight glycoproteins. TSP-1 and TSP-2 are homotrimeric molecules, composed of three identical disulfide-linked polypeptide chains, while TSP-3, TSP-4 and COMP are homopentameric molecules (5 chains). TSPs are stored in the alpha-granules of platelets and secreted by a variety of mesenchymal and epithelial cells (Majack et al. (1987)Cell Membrane 3:57-77; Adams (1997) Int J Biochem Cell Biol 29(6):861-5). Platelets secrete TSPs when activated in the blood by such physiological agonists such as thrombin and collagen (Lawler, J. (1986) Blood 67:112-123). TSPs have lectin properties and a broad function in the regulation of fibrinolysis and as a transient component of the extracellular matrix (ECM), and are one of a group of ECM proteins which have adhesive properties. TSPs bind to fibronectin and fibrinogen (Lahav et al. (1984) Eur J Biochem 145:151-6).

TSPs have also been implicated in the response of cells to growth factors. Submitogenic doses of PDGF induce a rapid but transitory increase in TSP synthesis and secretion by rat aortic smooth muscle cells (Majack et al. (1985) J Biol Chem 101:1059-70). PDGF responsiveness to TSP synthesis in glial cells has also been shown (Asch et al. (1986) Proc Natl Acad Sci 83:2904-8). TSP mRNA levels rise rapidly in response to PDGF (Majack et al. (1987) J Biol Chem 262:8821-5). TSPs act synergistically with epidermal growth factor to increase DNA synthesis in smooth muscle cells (Majack et al. (1986), Proc Natl Acad Sci 83:9050-4), and monoclonal antibodies to TSPs inhibit smooth muscle cell proliferation (Majack et al. J Biol Chem 106:415-22 (1988)). TSPs modulate local adhesions in endothelial cells, and TSPs, particularly TSP-1 primarily derived from platelet granules, are known to be an important activator of transforming growth factor beta-1 (TGFB-1) (Crawford et al., (1998) Cell 93:1159) and appear to be a potential link between platelet-thrombosis and development of atherosclerosis.

Current management of CVD utilizes molecular markers for cholesterol, clotting factors, homocysteine and other gene products which aid in the diagnosis and monitoring of disease progression. There is a need to identify new biomarkers that correlate with cardiovascular disease and provide physicians with new and more accurate tools upon which to base medical decisions regarding the diagnosis and monitoring of cardiovascular disease. The present invention satisfies these needs.

### SUMMARY OF THE INVENTION

The invention relates to the discovery of an important correlation between the level of thrombospondin (referred to herein as TSP) in blood fluids, and cardiovascular disease. In particular, it has been discovered that there is a correlation between the level of TSP (e.g. TSP-1 and TSP-4) protein and previously identified single nucleotide polymorphisms (SNPs) in TSP genes (e.g., TSP-1, TSP-2, and TSP-4) associated with cardiovascular disease. The level of TSP protein and/or TSP gene expression may therefore be used as a marker for cardiovascular disease.

In one aspect, the invention relates to a method of diagnosing or aiding in the diagnosis of a cardiovascular disease in a patient. The method includes comparing the level of a thrombospondin marker in a sample and the normal level of expression of the thrombospondin marker in a control, non-cardiovascular disease sample, where a significant difference between the level of thrombospondin marker in the patient sample and the normal thrombospondin marker level is an indication that the patient is afflicted with cardiovascular disease. In another aspect, the invention provides a method for predicting the likelihood that an individual will or will not have a cardiovascular disease, (e.g., whether a patient is predisposed to cardiovascular disease). The method includes comparing the level of a thrombospondin marker in a sample and the normal level of expression of a thrombospondin marker in a control non-cardiovascular disease sample. A significantly different level of the thrombospondin marker in the sample, relative to the normal level, is an indication that the patient is at risk to develop cardiovascular disease.

In yet another aspect, the invention provides a method for monitoring the progression of cardiovascular disease in a patient. The method includes:

a) detecting in a patient sample at a first point in time, the level of a thrombospondin marker,

b) repeating step a) at a subsequent point in time, and

c) comparing the level of thrombospondin marker detected in steps a) and b),

and therefrom monitoring the progression of cardiovascular disease in the patient. In one embodiment, between the first point in time and the subsequent point in time the patient has undergone treatment for cardiovascular disease (e.g., anticoagulant therapy).

In still another aspect, the invention provides a method of assessing the efficacy of a compound for inhibiting cardiovascular disease in a patient. The method includes comparing the level of a thrombospondin marker in a first sample obtained from the patient and maintained in the presence of the compound, and the level of a thrombospondin marker in a second sample obtained from the patient and maintained in the absence of the compound, wherein a significantly enhanced level of a thrombospondin marker in the first sample, relative to the second sample, is an indication that the compound is efficacious for inhibiting cardiovascular disease in the patient. In one embodiment, the first and second samples are portions of a single sample obtained from the subject. In a further embodiment, the first and second samples are portions of pooled samples obtained from the subject.

In a further aspect, the invention provides a method of assessing the efficacy of a therapy for inhibiting cardiovascular disease in a patient. The method includes comparing the level of a thrombospondin marker in the first sample obtained from the patient prior to providing at least a portion of the therapy to the patient, and the level of a thrombospondin marker in a second sample obtained from the patient following provision of the portion of the therapy, wherein a significantly enhanced level of a thrombospondin marker in the second sample, relative to the first sample, is an indication that the therapy is efficacious for inhibiting cardiovascular disease in the patient. In one embodiment, the therapy is anticoagulant therapy.

In another aspect, the invention provides a kit for diagnosing or aiding in the diagnosis of a cardiovascular disease. The kit includes reagents for assessing the level of a thrombospondin marker. In another aspect, the invention provides a kit for assessing the suitability of a compound for inhibiting cardiovascular disease in a patient. The kit includes the compound and a reagent for assessing expression of a thrombospondin marker.

In another aspect, the invention relates to pharmaceutical compositions comprising a TSP gene or gene product or active portion thereof preferably a TSP-1, TSP-2 and/or TSP-4 gene or gene product, or active portion thereof, for use in the treatment of cardiovascular diseases. The invention further relates to the use of agonists and antagonists of TSP activity, preferably TSP-1, TSP-2, and TSP-4 activity for use in the treatment of cardiovascular diseases. In one embodiment of the methods of the present invention, the patient sample is a blood fluid, e.g., whole blood, blood serum, blood having platelets removed therefrom and plasma. In another embodiment, the sample is obtained from a human subject.

In yet another embodiment of the invention, the thrombospondin marker is selected from the group consisting of a TSP protein or fragment thereof, preferably a TSP-1 protein, a TSP-2 protein, a TSP-4 protein, a fragment of a TSP-1 protein, a fragment of a TSP-2 protein, or a fragment of a TSP-4 protein. In still another embodiment, the presence of the marker is detected using a reagent which specifically binds with a TSP protein or a fragment thereof. In a preferred embodiment, the reagent is selected from the group consisting of an antibody, an antibody derivative, and an antibody fragment.

In a further embodiment, the thrombospondin marker is selected from the group consisting of a TSP nucleic acid molecule, preferably a TSP-1 nucleic acid molecule, a TSP-2 nucleic acid molecule, or a TSP-4 nucleic acid molecule.

In a particular embodiment the cardiovascular disease is selected from the group consisting of atherosclerosis, coronary artery disease (CAD), myocardial infarction (MI), stroke, peripheral vascular diseases, venous thromboembolism and pulmonary embolism. In a preferred embodiment, the cardiovascular disease is selected from the group consisting of CAD and MI.

The methods of the present invention are particularly useful for patients having an enhanced risk of developing cardiovascular disease (e.g., patients having a familial history of cardiovascular disease and patients who are at least about 50 years of age).

### FIGURES

FIGS. 1A-1D depict the specific reference nucleotide sequence (SEQ ID NO: 1) and amino acid sequence (SEQ ID NO: 2) for TSP-1.

FIGS. 2A-2C depict the specific reference nucleotide sequence (SEQ ID NO:3) and amino acid sequence (SEQ ID NO:4) for TSP-2.

FIGS. 3A-3C depict the specific reference nucleotide sequence (SEQ ID NO:5) and amino acid sequence (SEQ ID NO:6) for TSP-4.

FIG. 4 is a graph depicting correlation between repeated measures of plasma thrombospondin (r²=0.96).

### DETAILED DESCRIPTION OF THE INVENTION

The invention relates to a newly discovered correlation between levels of thrombospondin (e.g., TSP-1 and/or TSP-4) protein and expression of TSP genes and cardiovascular disease. In particular, it has been found that the level of TSP protein in blood fluids correlates with the presence of previously identified single nucleotide polymorphisms (SNPs) within TSP genes which have been correlated with cardiovascular disease in a subject. Compositions and methods are therefore provided for detecting the presence of cardiovascular disease in a subject, the stage or severity of a cardiovascular disease, and other characteristics of cardiovascular disease that are relevant to prevention, diagnosis, characterization, and therapy of cardiovascular disease in a patient.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “cardiovascular disease” (CVD) is any disease or disorder that affects the cardiovascular system. A cardiovascular disease or disorder includes, but is not limited to atherosclerosis, coronary heart disease or coronary artery disease (CAD), myocardial infarction (MI), stroke, peripheral vascular diseases, venous thromboembolism, and pulmonary embolism.

A “thrombospondin marker” or “TSP marker” of the invention is a TSP protein or a TSP nucleic acid molecule. A TSP marker therefore includes a polymer corresponding to at least one of the nucleic acid sequences set forth in SEQ ID NO:1, SEQ ID NO:3, or SEQ ID NO:5, or a fragment of the sequence, or the amino acid sequence set forth in SEQ ID NO: 2, SEQ ID NO: 4 or SEQ ID NO: 6, or a fragment of the sequence. In particular, a TSP marker of the invention may be a TSP protein comprising the amino acid sequence listed in SEQ ID NO: 2, SEQ ID NO: 4 or SEQ ID NO: 6, or a fragment of the sequence. A TSP marker may also be a TSP nucleic acid molecule comprising a nucleotide sequence listed in SEQ ID NO:1, SEQ ID NO:3, or SEQ ID NO:5, or a fragment of the sequence, or a sequence which hybridizes under high stringency conditions with a nucleotide sequence listed in SEQ ID NO:1, SEQ ID NO:3, or SEQ ID NO:5, or a fragment of the sequence. TSP nucleic acid molecules include, without limitation, sense and anti-sense strands of genomic DNA (i.e. including any introns occurring therein), and RNA generated by transcription of genomic DNA.

The “normal” level of a TSP protein is the level of a TSP protein in a subject, e.g., a human, not afflicted with or predisposed to cardiovascular disease. Likewise, the “normal” level of a TSP nucleic acid molecule is the level of expression of the TSP nucleic acid molecule in a subject, e.g. a human, not afflicted with or predisposed to cardiovascular disease. A “control non-cardiovascular disease sample” refers to a sample from a subject, e.g., a human, not affected with or predisposed to cardiovascular disease.

The term “polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair, in which case it is referred to as a single nucleotide polymorphism (SNP). The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of the population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. A genetic variant is a gene containing an altered, or polymorphic, base at the polymorphic site.

“Over-expression” and “under-expression” of a thrombospondin gene refers to expression of the gene in a patient at a greater or lesser level, respectively, than normal level of expression of the gene (e.g. at least two-fold greater or lesser level).

“Homologous” refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A “naturally-occurring” nucleic acid molecule refers to an RNA or DNA molecule having a nucleotide sequence that occurs in nature (e.g. encodes a natural protein).

The level of a TSP marker in a patient is “significantly” higher or lower than the normal level of a TSP marker if the level of TSP marker is greater or less, respectively, than the normal level by an amount greater than the standard error of the assay employed to assess the TSP marker and preferably at least twice, and more preferably three, four, five or ten times that amount. Alternately, the TSP marker level in the patient can be considered “significantly” higher or lower than the normal TSP marker level if the level of TSP marker is at least about two, and preferably at least about three, four, or five times, higher or lower, respectively, than the normal the level of TSP marker in a sample.

Cardiovascular disease is “inhibited” if at least one symptom of the specific cardiovascular disease is alleviated, terminated, slowed, or prevented.

A “kit” is any manufacture (e.g a package or container) comprising at least one reagent, e.g. an antibody or a probe, for specifically detecting a TSP marker (e.g., a TSP protein level or TSP gene expression level) in a sample. The manufacture may be promoted, distributed, or sold as a unit for performing the methods of the present invention.

Description

The present invention is based, in part on identification of correlation between thrombospondin protein levels and/or thrombospondin gene expression levels of members of the thrombospondin (TSP) family (e.g. TSP-1, and TSP-4) and cardiovascular disease. Cardiovascular disease includes but is not limited to, atherosclerosis, premature coronary artery disease (CAD) (or coronary heart disease), myocardial infarction (MI), stroke, peripheral vascular diseases, venous thromboembolism and pulmonary embolism. In a preferred embodiment, the cardiovascular disease is selected from the group consisting of CAD and MI. TSP protein level and/or TSP gene expression therefore serve as markers for cardiovascular disease and risk for cardiovascular disease.

Genetic variants which are significantly correlated with cardiovascular disease have been identified by the analysis of DNA from 240 patients with MI or coronary revascularization before age 45 (men) or 50 (women) and 422 general population controls. Cases were drawn (one per family) from a retrospective collection of sibling pairs with premature CAD. Controls were ascertained through random-digit dialing. Both cases and controls were Caucasian. A complete database of phenotypic and laboratory variables for the affected patients afforded logistic regression to control for age, diabetes, body mass index, gender.

Protein levels of TSP-1, TSP-2, and TSP-4 in plasma were analyzed from the same sample of 240 patients with the same cases and controls, as was used to identify the genetic variants. Significant correlations were found between the TSP-1 and TSP-4 protein levels in patient plasma and the presence of the previously identified SNPs (see Example 1), which have been correlated with cardiovascular disease. The level of TSP-1 plasma protein was associated with the TSP-1 asparagine/serine (N/S) genotype (p=0.1). The level of TSP-4 plasma protein was associated with the TSP-4 alanine/proline (A/P) genotype (p=0.10) (see Example 2). There therefore exists a significant association between the protein levels of TSP-1 and a suggestive association for TSP-4, the presence of at least one copy of a variant allele, and cardiovascular disease. The lack of a significant association between plasma levels and the variant TSP-2 does not indicate a lack of correlation between genotype and plasma level of TSP, but rather may be due to the specificity of the antibodies used to detect the TSP-2 protein.

Furthermore, for both TSP-1 and TSP-4, the genotypes associated with the highest risk of MI also have the lowest levels of thrombospondin. Therefore, low levels of TSP may be correlated with increased risk of MI.

The invention thus relates to a method for predicting the likelihood that an individual will or will not have a cardiovascular disease, or for aiding in the diagnosis of a cardiovascular disease, or predicting the likelihood of having altered symptomology associated with a cardiovascular disease.

In a particular embodiment, the individual is an individual who has or is at risk to develop a cardiovascular disease. In another embodiment the individual exhibits clinical symptomology associated with a cardiovascular disease. In yet another embodiment, the individual has been clinically diagnosed as having a cardiovascular disease.

The level of TSP marker level in a sample (e.g., a blood fluid sample) from a subject is herein correlated with cardiovascular disease in the subject. The invention thus includes compositions, kits, and methods for assessing cardiovascular disease. The compositions, kits, and methods of the invention have the following uses, among others:

1) assessing whether a subject is afflicted with cardiovascular disease:

2) assessing whether a subject is predisposed to cardiovascular disease;

3) assessing the stage of cardiovascular disease in a subject;

4) assessing the efficacy of one or more compounds for inhibiting cardiovascular disease in a patient;

5) assessing the efficacy of a therapy for inhibiting cardiovascular disease in a subject;

6) monitoring the progression of cardiovascular disease in a patient;

7) selecting a composition or therapy for inhibiting cardiovascular disease in a patient;

8) treating a patient afflicted with cardiovascular disease;

9) inhibiting cardiovascular disease in a patient;

10) inhibiting a cardiovascular disease in a subject at risk for developing cardiovascular disease.

The invention thus includes a method of assessing whether a subject is afflicted with cardiovascular disease. This method comprises comparing, for example, the thrombospondin (e.g., TSP-1, TSP-2, or TSP-4) protein and/or TSP gene expression level in a patient sample (e.g., a blood fluid sample) and the normal level of the TSP protein and/or TSP gene expression in a control, e.g., a normal or non-cardiovascular disease sample. A significant difference between the TSP protein level and/or TSP gene expression level present in the patient sample and the normal level is an indication that the patient is afflicted with cardiovascular disease.

Examples of blood fluids include whole blood, blood serum, blood having platelets removed therefrom (e.g., plasma), and platelets. In these embodiments, the level of TSP protein can be measured by assessing the amount (e.g., absolute amount or concentration) of the protein in a blood fluid, e.g., blood serum, obtained from a patient. The level of TSP gene expression can be measured by assessing the amount (e.g., absolute amount or concentration) of TSP nucleic acid molecules in blood fluid (e.g., platelets) obtained from a patient. The fluid can, of course, be subjected to a variety of well-known post-collection preparative and storage techniques (e.g. storage. freezing, ultrafiltration, concentration, evaporation, centrifugation, etc.) prior to assessing the amount of TSP protein or nucleic acid in the fluid.

The level of TSP protein or expression of a TSP gene may be assessed by any of a wide variety of well known methods for detecting expression of protein or a transcribed molecule. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In a preferred embodiment, the level of a TSP protein or a fragment thereof is assessed using an antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a protein, e.g., a TSP protein such as TSP-1, TSP-2, or TSP-4, or fragments thereof. Examples of commercially available antibodies specific to TSP protein include, without limitation, P10 and P12 available from Coulter Corporation™.

In another preferred embodiment, expression of a TSP gene, or fragment thereof, is assessed by preparing mRNA/cDNA (i.e. a transcribed polynucleotide) from a patient sample, and by hybridizing the mRNA/cDNA with a reference polynucleotide which is a complement of a polynucleotide comprising TSP gene, and fragments thereof. cDNA can, optionally, be amplified using any of a variety of polymerase chain reaction methods prior to hybridization with the reference polynucleotide; preferably, it is not amplified. Expression of a TSP gene, or fragment thereof, can likewise be detected using quantitative PCR to assess the level of expression of the TSP gene(s). Alternatively, any of the many known methods of detecting mutations or variants (e.g., single nucleotide polymorphisms, deletions, etc.) of a TSP gene of the invention may be used to detect occurrence of a TSP gene in a patient.

In a related embodiment, a mixture of transcribed TSP polynucleotides obtained from the sample is contacted with a substrate having fixed thereto a TSP polynucleotide complementary to or homologous with at least a portion (e.g., at least 7, 10, 15, 20, 25, 30, 40, 50, 100, 500, or more nucleotide residues) of a TSP polynucleotide used in the methods of the invention. If TSP polynucleotides complementary to or homologous with are differentially detectable on the substrate (e.g., detectable using different chromophores or fluorophores, or fixed to different selected positions), then the levels of expression of TSP genes, or fragments thereof, can be assessed simultaneously using a single substrate (e.g., a “gene chip” microarray of polynucleotides fixed at selected positions). When a method of assessing TSP expression is used which involves hybridization of one nucleic acid with another, it is preferred that the hybridization be performed under stringent hybridization conditions.

Because the compositions, kits, and methods of the invention rely on detection of a difference in the levels of one or more TSP markers of the invention, it is preferable that the level of the marker is significantly greater than the minimum detection limit of the method used to assess the marker level in a normal sample.

It is recognized that the compositions, kits, and methods of the invention will be of particular utility to patients having an enhanced risk of developing cardiovascular disease and their medical advisors. Patients recognized as having an enhanced risk of developing cardiovascular disease include, for example, patients having a familial history of cardiovascular disease, hypertension, obesity, older age, and patients who smoke.

The level of a TSP marker (e.g., a TSP protein or TSP nucleic acid molecule) in normal blood fluid samples (i.e,. blood fluid samples from a subject who is free from cardiovascular disease) can be assessed in a variety of ways. Alternately, and particularly as further information becomes available as a result of routine performance of the methods described herein, population-average values for normal TSP protein levels and/or expression levels of the TSP nucleic acid molecules used in the methods of the invention may be used. In other embodiments, the ‘normal’ level of a TSP marker may be determined by assessing the level of a TSP protein and/or TSP gene expression in a patient sample obtained from a non-cardiovascular disease-afflicted patient, from a patient sample obtained from a patient before the suspected onset of cardiovascular disease in the patient, from archived patient samples, and the like.

The invention includes a kit for assessing the presence of cardiovascular disease in a subject (e.g., in a human subject). The kit may comprise a reagent or a plurality of reagents, each of which is capable of binding specifically with a TSP marker (e.g., TSP protein and nucleic acid molecule). Suitable reagents for binding with a TSP polypeptide or protein include antibodies, antibody derivatives, antibody fragments, and the like. Suitable reagents for binding with a TSP nucleic acid molecule (e.g., a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, a normal sample of blood fluid, and the like.

The invention also includes a method of making an isolated hybridoma which produces an antibody useful for assessing whether a patient is afflicted with a cardiovascular disease. In this method, a TSP protein or a fragment thereof is isolated (e.g., by purification from a cell in which it is expressed or by transcription and translation of a nucleic acid encoding the protein in vivo or in vitro using known methods). A vertebrate, preferably a mammal such as a mouse, rat, rabbit, or sheep, is immunized using the isolated protein or fragment thereof. The vertebrate may optionally (and preferably) be immunized at least one additional time with the isolated protein or fragment, so that the vertebrate exhibits a robust immune response to the protein. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or fragment. The invention also includes hybridomas made by this method and antibodies made using such hybridomas. An antibody of the invention may also be used as a therapeutic agent for treating cardiovascular disease.

The invention also includes a method of assessing the efficacy of a compound for inhibiting cardiovascular disease. As described above, differences in the level of TSP markers (e.g., TSP proteins and TSP nucleic acid molecules) correlate with the presence of generic variants which are correlated with cardiovascular disease. Compounds which inhibit a cardiovascular disease in a patient will cause the level of a TSP protein and/or the expression of a TSP gene to change to a level nearer the normal level of expression (i.e., the level of a TSP protein and/or the expression of a TSP gene in a normal sample).

This method thus comprises comparing the level of TSP marker in a first sample (e.g, a blood fluid sample) maintained in the presence of a compound and the level of TSP marker in a second sample maintained in the absence of a compound. A significant decrease in the level of a TSP marker (e.g., TSP protein or TSP gene expression) is an indication that the compound inhibits cardiovascular disease. In one embodiment, the samples are blood fluid samples obtained from a patient and a plurality of compounds known to be effective for inhibiting various cardiovascular diseases are tested in order to identify the compound which is likely to best inhibit the cardiovascular disease in the patient.

This method may likewise be used to assess the efficacy of a therapy for inhibiting cardiovascular disease in a patient. In this method, the level of TSP marker in a pair of samples (one from a subject who has been subjected to the therapy, the other from a subject who has not been subjected to the therapy) is assessed. As with the method of assessing the efficacy of compounds, if the therapy induces a significant increase in the level of a TSP marker, then the therapy is efficacious for inhibiting cardiovascular disease in the subject. As above, if samples from a selected patient are used in this method, then alternative therapies can be assessed in vitro in order to select a therapy most likely to be efficacious for inhibiting cardiovascular disease in the patient.

Various aspects of the invention are described in further detail in the following subsections.

I. Isolated Proteins and Antibodies

One aspect of the invention pertains to isolated thrombospondin proteins (e.g., TSP-1, TSP-2, and TSP-4), and biologically active portions thereof, as well as polypeptide fragments suitable for use as immunogens to raise antibodies directed against a TSP polypeptide. SEQ ID NOS: 2, 4 and 6 set forth the amino acid sequences for TSP-1, TSP-2 and TSP-4, respectively.

In one embodiment, a native TSP polypeptide can be isolated from cells or tissue sources by an appropriate purification scheme using standard protein purification techniques. In another embodiment, TSP polypeptides are produced by recombinant DNA techniques. Alternative to recombinant expression, a TSP polypeptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a TSP polypeptide include polypeptides which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted can be prepared by recombinant techniques and evaluated for one or more of the functional activities of the native form of a polypeptide of the invention.

Preferred polypeptides have the amino acid sequence listed in SEQ ID NO:2, SEQ ID NO:4, or SEQ ID NO:6. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score =100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a TSP protein used in the methods of the invention. As used herein, a “chimeric protein” or “fusion protein” comprises all or part (preferably a biologically active part) of a polypeptide corresponding to a TSP protein used in the methods of the invention operably linked to a heterologous polypeptide (i.e., a polypeptide other than the polypeptide corresponding to the TSP protein). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a TSP protein used in the methods of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to TSP protein used in the methods of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a TSP protein used in the methods of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a TSP polypeptide used in the methods of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the TSP polypeptides corresponding to the SNPs described herein (i.e., G334u4 and G355u2). Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a TSP polypeptide used in the methods of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a TSP protein used in the methods of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a TSP protein used in the methods of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide used in the methods of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:104 1-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Antibodies used in the methods of the invention may be used as therapeutic agents in treating cardiovascular disease. In a preferred embodiment, completely human TSP antibodies used in the methods of the invention are used for therapeutic treatment of human cardiovascular disease patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a TSP protein used in the methods of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a TSP polypeptide (e.g., a monoclonal antibody) can be used to isolate the polypeptide by standard techniques, such as affinity chromatography or immunoprecipitation. Moreover, such an antibody can be used to detect a TSP polypeptide (e.g., in a cellular lysate or cell supernatant) in order to evaluate the level and pattern of expression of the TSP gene. The antibodies can also be used diagnostically to monitor protein levels in tissues or body fluids (e.g., blood fluid such as plasma) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified TSP antibodies or fragments thereof, and non-human antibodies or fragments thereof, which antibodies or fragments specifically bind to a polypeptide comprising an amino acid sequence selected from the group consisting of the TSP amino acid sequences, an amino acid sequence encoded by the cDNA of the present invention, a fragment of at least 15 amino acid residues of an amino acid sequence of a TSP molecule, an amino acid sequence which is at least 95% identical to a TSP amino acid sequence (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. In various embodiments the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of the present invention, an amino acid sequence encoded by the cDNA of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention. or a complement thereof, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of a TSP amino acid sequence.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes a TSP polypeptide, the method comprising immunizing a mammal with a polypeptide. The polypeptide used as an immungen comprises an amino acid sequence selected from the group consisting of the amino acid sequence of the present invention, an amino acid sequence encoded by the cDNA of the nucleic acid molecules of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C.

After immunization, a sample is collected from the mammal that contains an antibody that specifically recognizes the polypeptide. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

II. Isolated Nucleic Acid Molecules

One aspect of the invention pertains to isolated TSP nucleic acid molecules used in the methods of the invention. SEQ ID NOS: 1, 3 and 5 set forth the nucleotide sequences for TSP-1, TSP-2 and TSP-4, respectively. Isolated TSP nucleic acid molecules used in the methods of the invention also include TSP nucleic acid molecules sufficient for use as hybridization probes to identify nucleic acid molecules that correspond to a TSP molecule of the invention, including TSP nucleic acids which encode a TSP polypeptide corresponding to a TSP nucleic acid molecule used in the methods of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule comprises a protein-coding sequence and is free of sequences which naturally flank the coding sequence in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule used in the methods of the present invention, e.g., a TSP nucleic acid encoding a TSP polypeptide, can be isolated using standard molecular biology techniques and the sequence information in the database records described herein. Using all or a portion of such nucleic acid sequences, nucleic acid molecules used in the methods of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A TSP nucleic acid molecule used in the methods of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule used in the methods of the invention comprises a TSP nucleic acid molecule which has a nucleotide sequence complementary to the nucleotide sequence of a TSP nucleic acid used in the methods of the invention or to the nucleotide sequence of a TSP nucleic acid encoding a TSP used in the methods of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a TSP nucleic acid molecule used in the methods of the invention can comprise only a portion of a TSP nucleic acid sequence, wherein the full length nucleic acid sequence comprises a marker of the invention or which encodes a polypeptide corresponding to a marker of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a TSP nucleic acid molecule used in the methods of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of TSP nucleic acids encoding a TSP protein used in the methods of the invention, and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 0.1-0.5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid corresponding to a TSP nucleic acid encoding a TSP protein used in the methods of the invention. As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6×sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, August 1996:1675-1680; Breslauer, et al., Proc. Natl. Acad. Sci. USA, Volume 83, June 1986:3746-3750; Van Ness, et al., Nucleic Acids Research, Volume 19, No. 19, September 1991:5143-5151; McGraw, et al., BioTechniques, Volume 8, No. 6 1990: 674-678; and Milner, et al., Nature Biotechnology, Volume 15, June 1997:537-541, all expressly incorporated by reference.

In addition to naturally-occurring allelic variants of a nucleic acid molecule used in the methods of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide used in the methods of the invention that contain changes in amino acid residues that are not essential for activity. Such polypeptides differ in amino acid sequence from the naturally-occurring TSP used in the methods of the invention, yet retain biological activity. In one embodiment, such a protein has an amino acid sequence that is at least about 40% identical, 50%, 60%, 70%, 80%, 90%, 95%, or 98% identical to the amino acid sequence of one of the TSP proteins used in the methods of the invention.

An isolated nucleic acid molecule encoding a variant TSP protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, teucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules. i.e., molecules which are complementary to a sense TSP nucleic acid used in the methods of the invention, e.g., complementary to the coding strand of a double-stranded cDNA molecule corresponding to a TSP mRNA sequence used in the methods of the invention. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a TSP polypeptide used in the methods of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid used in the methods of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense TSP nucleic acid molecules used in the methods of the invention are typically administered to a subject or generated in situ such that they hybridize with or bind to cellular mRNA and/or genomic DNA encoding a TSP polypeptide used in the methods of the invention to thereby inhibit expression of TSP, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into an ovary-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the TSP nucleic acid molecules used in the methods of the invention can be modified at the base moiety sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon TSP nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

III. Pharmaceutical Compositions

The TSP proteins, nucleic acid molecules, and antibodies (also referred to herein as “active compounds”) used in the methods of the invention can be incorporated into pharmaceutical compositions suitable for administration. Such compositions typically comprise the protein nucleic acid molecule, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a TSP marker used in the methods of the invention. Such methods comprise formulating a pharmaceutically acceptable carrier with an agent which modulates expression or activity of a TSP marker used in the methods of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a TSP protein used in the methods of the invention and one or more additional active compounds.

The invention also provides methods (also referred to herein as “screening assays”) for identifying modulators, i.e., candidate or compounds or agents (e.g., peptides, peptidomimetics, peptoids, small molecules or other drugs) which (a) bind to a TSP marker used in the methods used in the methods, or (b) have a modulatory (e.g., stimulatory or inhibitory) effect on the activity of a TSP marker or, more specifically, (c) have a modulatory effect on the interactions of a TSP marker with one or more of its natural substrates (e.g., peptide, protein, hormone, co-factor, or nucleic acid), or (d) have a modulatory effect on the expression of a TSP marker. Such assays typically comprise a reaction between the TSP marker and one or more assay components. The other components may be either the compound itself, or a combination of compound and a natural binding partner of the TSP marker.

The compounds of the present invention may be obtained from any available source, including systematic libraries of natural and/or synthetic compounds, compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or compounds which are substrates of a TSP marker. In another embodiment, the invention provides assays for screening candidate or compounds which bind to a TSP marker. Determining the ability of the compound to directly bind to a TSP protein can be accomplished, for example, by coupling the compound with a radioisotope or enzymatic label such that binding of the compound to the TSP marker can be determined by detecting the labeled TSP marker compound in a complex. For example, compounds (e.g., TSP marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or compounds which modulate the activity of a TSP marker. In all likelihood, the TSP marker can, in vivo, interact with one or more molecules, such as but not limited to, peptides, proteins, hormones, cofactors and nucleic acids. For the purposes of this discussion, such cellular and extracellular molecules are referred to herein as “binding partners” or TSP marker “substrate ”.

One necessary embodiment of the invention in order to facilitate such screening is the use of the TSP marker to identify its natural in vivo binding partners. There are many ways to accomplish this which are known to one skilled in the art. One example is the use of the TSP marker as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the TSP marker, e.g., TSP protein (binding partners) and, therefore, are possibly involved in the natural function of the TSP markers. Such TSP markers binding partners are also likely to be involved in the propagation of signals by the TSP marker or downstream elements of a TSP marker-mediated signaling pathway. Alternatively, such TSP marker binding partners may also be found to be inhibitors of the TSP marker.

The two-hybrid system is based on the modular nature of most transcription factors, which consist of separable DNA-binding and activation domains. Briefly, the assay utilizes two different DNA constructs. In one construct, the gene that encodes a TSP protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TSP protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the TSP protein.

In a further embodiment, assays may be devised through the use of the invention for the purpose of identifying compounds which modulate (e.g., affect either positively or negatively) interactions between a TSP protein and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is a cardiovascular disease TSP protein identified herein, the known binding partner and/or substrate of same, and the compound. Compounds can be supplied from any source.

The basic principle of the assay systems used to identify compounds that interfere with the interaction between the TSP protein and its binding partner involves preparing a reaction mixture containing the TSP protein and its binding partner under conditions and for a time sufficient to allow the two products to interact and bind, thus forming a complex. In order to an agent for inhibitory activity, the reaction mixture is prepared in the presence and absence of the compound. The compound can be initially included in the reaction mixture, or can be added at a time subsequent to the addition of the TSP protein and its binding partner. Control reaction mixtures are incubated without the compound or with a placebo. The formation of any complexes between the TSP protein and its binding partner is then detected. The formation of a complex in the control reaction, but less or no such formation in the reaction mixture containing the compound, indicates that the compound interferes with the interaction of the TSP protein and its binding partner. Conversely, the formation of more complex in the presence of compound than in the control reaction indicates that the compound may enhance interaction of the TSP protein and its binding partner.

The assay for compounds that interfere with the interaction of the TSP protein with its binding partner may be conducted in a heterogeneous or homogeneous format. Heterogeneous assays involve anchoring either the TSP protein or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, compounds that interfere with the interaction between the TSP proteins and the binding partners (e.g., by competition) can be identified by conducting the reaction in the presence of the substance, i.e., by adding the substance to the reaction mixture prior to or simultaneously with the TSP protein and its interactive binding partner. Alternatively, compounds that disrupt preformed complexes, e.g., compounds with higher binding constants that displace one of the components from the complex, can be tested by adding the compound to the reaction mixture after complexes have been formed. The various formats are briefly described below.

In a heterogeneous assay system, either the TSP protein or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the TSP protein or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/TSP protein fusion proteins or glutathione-S-transferase/binding partner can be adsorbed onto glutathione sepharose beads (Sigma Chemical, St. Louis, Mo.) or glutathione derivatized microtiter plates, which are then combined with the compound or the compound and either the non-adsorbed TSP protein or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of TSP protein binding or activity determined using standard techniques.

Other techniques for immobilizing proteins on matrices can also be used in the screening assays of the invention. For example, either a TSP protein or a TSP protein binding partner can be immobilized utilizing conjugation of biotin and streptavidin. Biotinylated TSP protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the compound. The formed complexes are then separated from unreacted components, and the amount of complex formed is determined. As mentioned for heterogeneous assay systems, the order of addition of reactants to the liquid phase can yield information about which compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci August 1993;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a compound can be compared, thus offering information about the ability of the compound to modulate interactions between the TSP protein and its binding partner.

Also within the scope of the present invention are methods for direct detection of interactions between the TSP protein and its natural binding partner and/or a compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., TSP protein or compound) such that its emitted fluorescent energy will be absorbed by a fluorescent label on a second, ‘acceptor’ molecule (e.g., TSP protein or compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A substance which either enhances or hinders participation of one of the species in the preformed complex will result in the generation of a signal variant to that of background. In this way, substances that modulate interactions between a TSP protein and its binding partner can be identified in controlled assays.

In another embodiment, modulators of TSP expression are identified in a method wherein a cell is contacted with a candidate compound and the expression of TSP mRNA or protein, is determined. The level of expression of TSP mRNA or protein in the presence of the candidate compound is compared to the level of expression of TSP mRNA or protein in the absence of the candidate compound. The candidate compound can then be identified as a modulator of TSP expression based on this comparison. For example, when expression of TSP mRNA or protein is greater (statistically significantly greater) in the presence of the candidate compound than in its absence the candidate compound is identified as a stimulator of TSP mRNA or protein expression. Conversely, when expression of TSP mRNA or protein is less (statistically significantly less) in the presence of the candidate compound than in its absence the candidate compound is identified as an inhibitor of TSP mRNA or protein expression. The level of TSP mRNA or protein expression in the cells can be determined by methods described herein for detecting TSP mRNA or protein.

In another aspect, the invention pertains to a combination of two or more of the assays described herein. For example, a modulating agent can be identified using a cell-based or a cell free assay, and the ability of the agent to modulate the activity of a TSP protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an TSP modulating agent, an antisense TSP nucleic acid molecule, a TSP-specific antibody, or a TSP-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g., about 1 microgram per kilogram to about 500 milligrams per kilogram about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration. A method for lipidation of antibodies is described by Cruikshank et al. (1997) J. Acquired Immune Deficiency Syndromes and Human Retrovirology 14:193.

The nucleic acid molecules corresponding to a TSP gene of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

IV. Predictive Medicine

The present invention pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the level of a TSP marker, in order to determine whether an individual is at risk of developing cardiovascular disease. Such assays can be used for prognostic or predictive purposes to thereby prophylactically treat or modify risk behaviors prior to the onset of cardiovascular disease.

Yet another aspect of the invention pertains to monitoring the influence of agents (e.g., drugs or other compounds administered either to inhibit cardiovascular disease or to treat or prevent any other disorder {i.e. in order to understand any cardiovascular effects that such treatment may have }) on the level of a TSP a marker of the invention in clinical trials. These and other agents are described in further detail in the following sections.

A. Diagnostic Assays

An exemplary method for detecting a TSP marker in the methods of the invention in a biological sample involves obtaining a biological sample (e.g., a blood fluid sample) from a subject and contacting the biological sample with a compound or an agent capable of detecting the TSP marker (e.g., a TSP antibody, mRNA, genomic DNA, or cDNA). The detection methods of the invention can thus be used to detect TSP protein, mRNA, cDNA, or genomic DNA, for example, in a biological sample in vitro as well as in vivo. For example, in vitro techniques for detection of mRNA include Northern hybridizations and in situ hybridizations. In vitro techniques for detection of a TSP polypeptide used in the methods of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a TSP protein used in the methods of the invention include introducing into a subject a labeled antibody directed against the TSP protein. For example, the antibody can be labeled with a radioactive marker whose presence and location in a subject can be detected by standard imaging techniques.

A general principle of such diagnostic and prognostic assays involves preparing a sample or reaction mixture that may contain a TSP marker (e.g., a TSP-1, TSP-2, or TSP-4 protein or nucleic acid molecule) and a probe, under appropriate conditions and for a time sufficient to allow the TSP marker and probe to interact and bind, thus forming a complex that can be removed and/or detected in the reaction mixture. These assays can be conducted in a variety of ways.

For example, one method to conduct such an assay would involve anchoring a TSP marker or probe onto a solid phase support, also referred to as a substrate, and detecting target a TSP marker/probe complexes anchored on the solid phase at the end of the reaction. In one embodiment of such a method, a sample from a subject, which is to be assayed for presence and/or concentration of a TSP marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, TSP marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the TSP marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of TSP marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect TSP marker/probe complex formation without further manipulation or labeling of either component (TSP marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a TSP marker can be accomplished without labeling either assay component (probe or TSP marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with a TSP marker and probe as solutes in a liquid phase. In such an assay, the complexed TSP marker and probe are separated from uncomplexed components by any of a number of standard techniques, including but not limited to: differential centrifugation, chromatography, electrophoresis and immunoprecipitation. In differential centrifugation, TSP marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the TSP marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl Oct. 10, 1997;699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, TSP marker complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental embodiment set forth in Mullis 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the TSP marker, determinations may be based on the normalized expression level of the TSP marker. Expression levels are normalized by correcting the absolute expression level of a TSP marker by comparing its expression to the expression of a gene that is not a TSP marker, e.g., a housekeeping gene that is constitutively expressed. Suitable genes for normalization include housekeeping genes such as the actin gene, or epithelial cell-specific genes. This normalization allows the comparison of the TSP expression level in one sample, e.g., a patient sample, to another sample, e.g. a non-cardiovascular disease sample, or between samples from different sources.

Alternatively, the expression level can be provided as a relative expression level. To determine a relative expression level of a TSP marker, the level of expression of the TSP marker is determined for 10 or more samples of normal versus samples from a subject afflicted with a cardiovascular disease, preferably 50 or more samples, prior to the determination of the expression level for the sample in question. The mean expression level of each of the genes assayed in the larger number of samples is determined and this is used as a baseline expression level for the TSP protein or nucleic acid molecule. The expression level of the TSP marker determined for the sample (absolute level of expression) is then divided by the mean expression value obtained for that TSP marker. This provides a relative expression level.

Preferably, the samples used in the baseline determination will be from cardiovascular disease or from non-cardiovascular disease samples (e.g., blood fluid samples). Using expression found in samples from normal subjects as a mean expression score aids in validating whether the TSP marker assayed is cardiovascular disease specific. In addition, as more data is accumulated, the mean expression value can be revised, providing improved relative expression values based on accumulated data. Expression data from samples from subjects afflicted with cardiovascular disease provides a means for grading the severity of the cardiovascular disease state.

In another embodiment of the present invention, a TSP protein or fragment thereof is detected. A preferred agent for detecting a TSP protein or fragment thereof polypeptide used in the methods of the invention is an antibody capable of binding to a TSP protein or fragment thereof used in the methods of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Secreted proteins can be isolated from blood using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining the levels of TSP protein expression in blood fluid samples.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from blood fluids can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a TSP used in the methods of the invention in a biological sample (e.g. a blood fluid such as plasma). Such kits can be used to determine if a subject is suffering from or is at increased risk of developing cardiovascular disease. For example, the kit can comprise a labeled compound or agent capable of detecting a TSP marker used in the methods of the invention in a biological sample and means for determining the amount of the TSP marker (e.g., an antibody which binds TSP protein or an oligonucleotide probe which binds to a TSP nucleic acid molecule). Kits can also include instructions for interpreting the results obtained using the kit.

For antibody-based kits, the kit can comprise, for example: (1) a first antibody (e.g., attached to a solid support) which binds to a TSP protein or fragment thereof used in the methods of the invention; and, optionally, (2) a second, different antibody which binds to either the TSP protein or fragment thereof, or the first antibody and is conjugated to a detectable label.

B. Pharmacogenomics

Agents or modulators which have a stimulatory or inhibitory effect on level of a TSP marker used in the methods of the invention can be administered to individuals to treat (prophylactically or therapeutically) cardiovascular disease in the patient. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the level of a TSP marker used in the methods of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of a TSP marker used in the methods of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of level of a TSP marker used in the methods of the invention.

C. Monitoring Clinical Trials

Monitoring the influence of agents (e.g., drug compounds) on the level of a TSP marker used in the methods of the invention can be applied not only in basic drug screening, but also in clinical trials. For example, the effectiveness of an agent to affect marker expression can be monitored in clinical trials of subjects receiving treatment for cardiovascular disease. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of TSP marker in the pre-administration sample; (iii) obtaining one or more post-administration samples from the subject; (iv) detecting the level of TSP marker in the post-administration samples; (v) comparing the level of TSP marker in the pre-administration sample with the level of TSP marker in the post-administration sample or samples; and (vi) altering the administration of the agent to the subject accordingly. For example, increased administration of the agent can be desirable to increase or decrease TSP marker levels, i.e., to increase the effectiveness of the agent.

D. Surrogate Markers

The TSP markers of the invention may serve as surrogate markers for one or more disorders or disease states or for conditions leading up to disease states, and in particular, cardiovascular disease. As used herein, a “surrogate marker” is an objective biochemical marker which correlates with the absence or presence of a disease or disorder, or with the progression of a disease or disorder (e.g., with the presence or absence of a cardiovascular disease). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early cardiovascular disease, such as early coronary artery disease or arteriosclerosis), or when an assessment of disease progression is desired before a potentially dangerous clinical endpoint is reached (e.g., an analysis of HIV infection may be made using HIV RNA levels as a surrogate marker, well in advance of the undesirable clinical outcome of fully-developed AIDS). Examples of the use of surrogate markers in the art include: Koomen et al. (2000) J. Mass. Spectrom. 35: 258-264; and James (1994) AIDS Treatment News Archive 209.

The TSP markers used in the methods of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of a TSP marker in a subject, a drug or course of treatment may be selected that is optimized for the treatment of the specific disease likely to be present in the subject. Similarly, the presence or absence of a specific TSP marker level may correlate with drug response. The use of pharmacogenomic markers therefore permits the application of the most appropriate treatment for each subject without having to administer the therapy.

E. Electronic Apparatus Readable Media and Arrays

Electronic apparatus readable media comprising a TSP marker of the present invention is also provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the TSP markers of the present invention.

A variety of software programs and formats can be used to store the marker information of the present invention on the electronic apparatus readable medium. For example, the nucleic acid sequence corresponding to the TSP markers can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the TSP markers of the present invention.

By providing the TSP markers of the invention in readable form, one can routinely access the marker sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a cardiovascular disease or a pre-disposition to a cardiovascular disease, wherein the method comprises the steps of determining the presence or absence of a TSP marker and based on the presence or absence of the TSP marker, determining whether the subject has a cardiovascular disease or a pre-disposition to a cardiovascular disease and/or recommending a particular treatment for the cardiovascular disease or pre-cardiovascular disease condition.

The present invention further provides in an electronic system and/or in a network, a method for determining whether a subject has a cardiovascular disease or a pre-disposition to a cardiovascular disease associated with a TSP marker wherein the method comprises the steps of determining the presence or absence of the TSP marker and based on the presence or absence of the TSP marker, determining whether the subject has a cardiovascular disease or a pre-disposition to a cardiovascular disease, and/or recommending a particular treatment for the cardiovascular disease or pre-cardiovascular disease condition. The method may further comprise the step of receiving phenotypic information associated with the subject and/or acquiring from a network phenotypic information associated with the subject.

The present invention also provides in a network, a method for determining whether a subject has a cardiovascular disease or a pre-disposition to a cardiovascular disease associated with a TSP marker, said method comprising the steps of receiving information associated with the TSP marker receiving phenotypic information associated with the subject, acquiring information from the network corresponding to the TSP marker and/or cardiovascular disease, and based on one or more of the phenotypic information, the TSP marker, and the acquired information, determining whether the subject has a cardiovascular disease or a pre-disposition to a cardiovascular disease. The method may further comprise the step of recommending a particular treatment for the cardiovascular disease or pre-cardiovascular disease condition.

The present invention also provides a business method for determining whether a subject has a cardiovascular disease or a pre-disposition to a cardiovascular disease, said method comprising the steps of receiving information associated with the TSP marker, receiving phenotypic information associated with the subject, acquiring information from the network corresponding to the TSP marker and/or cardiovascular disease, and based on one or more of the phenotypic information, the TSP marker, and the acquired information, determining whether the subject has a cardiovascular disease or a pre-disposition to a cardiovascular disease. The method may further comprise the step of recommending a particular treatment for the cardiovascular disease or pre-cardiovascular disease condition.

The invention also includes an array comprising a TSP marker of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of cardiovascular disease, progression of cardiovascular disease, and processes, such a cellular transformation associated with cardiovascular disease.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention

This invention is further illustrated by the following examples which should not be construed as limiting. The contents of all references, patents and published patent applications cited throughout this application, as well as the Figures, are incorporated herein by reference.

### EXAMPLES

### Example 1

Identification of Single Nucleotide Polymorphisms in Thrombospondin Genes

To determine pivotal genes associated with premature coronary artery disease, DNA from 347 patients with MI or coronary revascularization before age 40 (men) or 45 (women) and 422 general population controls were analyzed. Cases were drawn (one per family) from a retrospective collection of sibling pairs with premature CAD. Controls were ascertained through random-digit dialing. Both cases and controls were Caucasian. A complete database of phenotypic and laboratory variables for the affected patients afforded logistic regression to control for age, diabetes, body mass index, and gender.

In particular, SNPs in TSP-1, TSP-2 and TSP-4 have been correlated with cardiovascular disease and, in particular, with premature CAD and MI. For CAD, 148 of 347 patients carried at least one copy of the TSP-4 variant compared with 142 of 422 control subjects. For premature MI, the association was even stronger, 91 of 187 cases vs. 142 of 422 controls had the variant. The TSP-1 SNP was rare; nonetheless, homozygosity for the variant allele gave an adjusted odds ratio of 9,5,p=0.04 for CAD and 10.1, p=0.05 for MI. For the TSP-2 variant, 87 of 177 patients with MI carried one copy of the TSP-2 variant and showed an increased risk of MI, although not statistically significant (OR=1.36, p=10). Individuals who carried two copies of the variant allele (homozygotes) were protected from MI (OR=0.38, p=0.03). Taken together these data suggest that genetic variants in the TSP-2 gene correlate with MI.

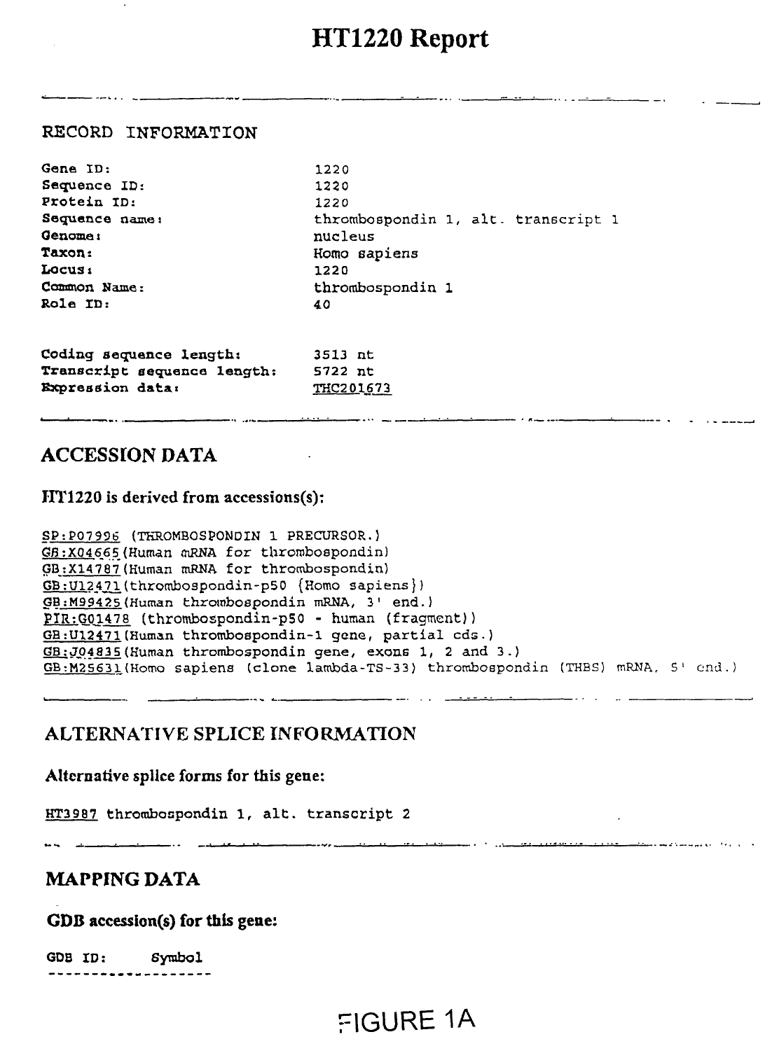

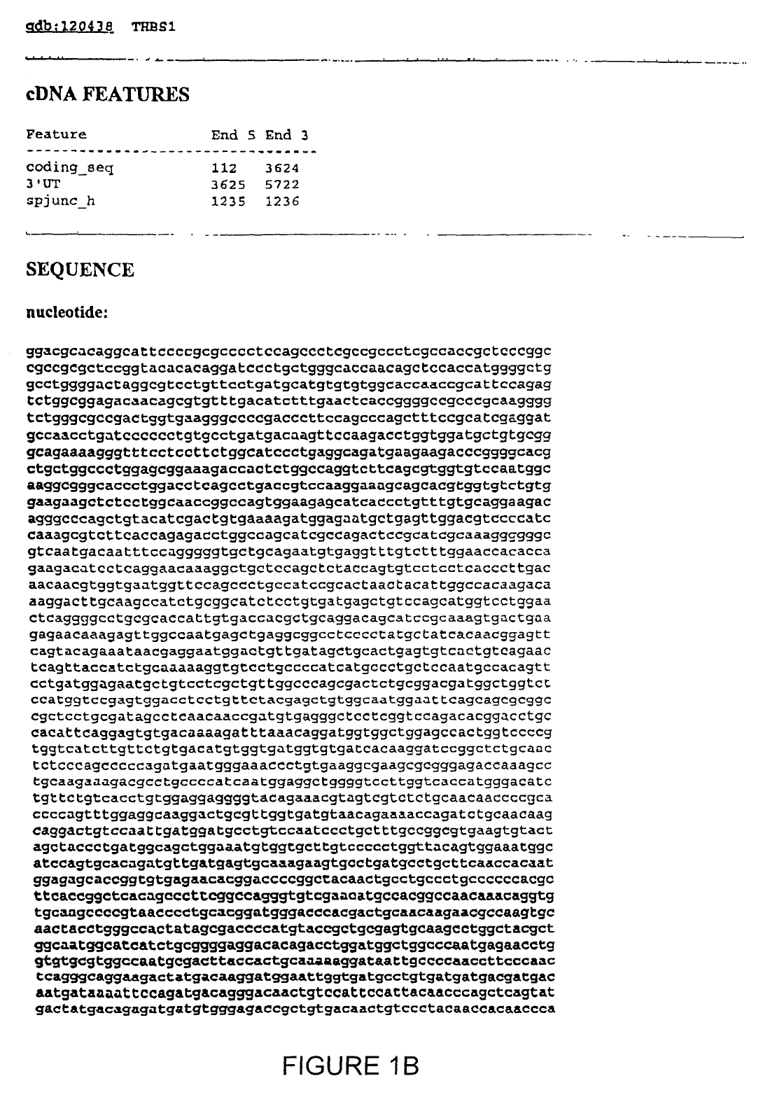

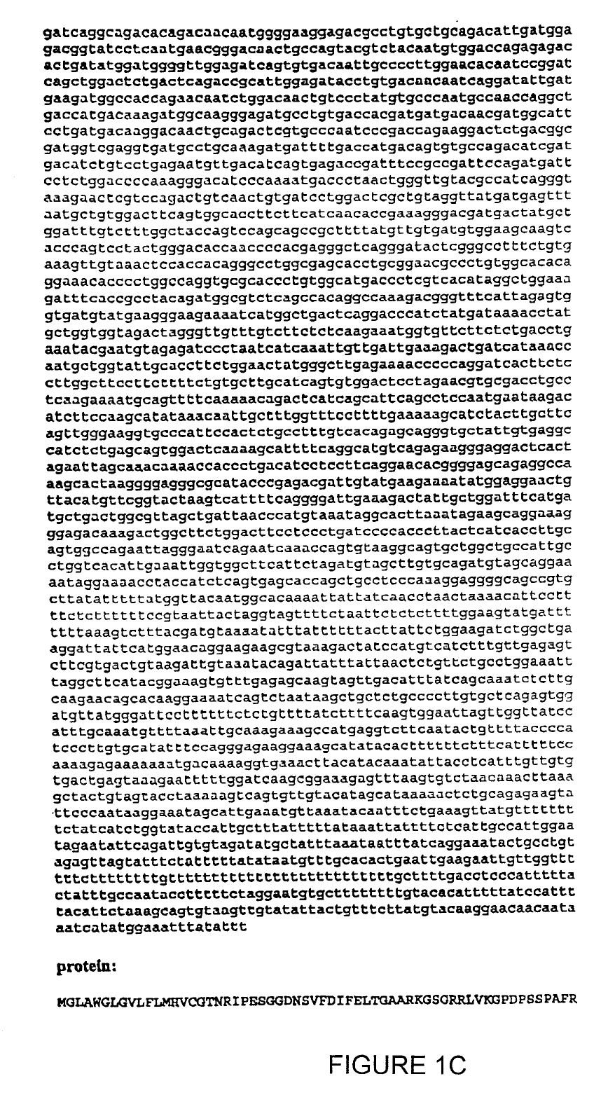

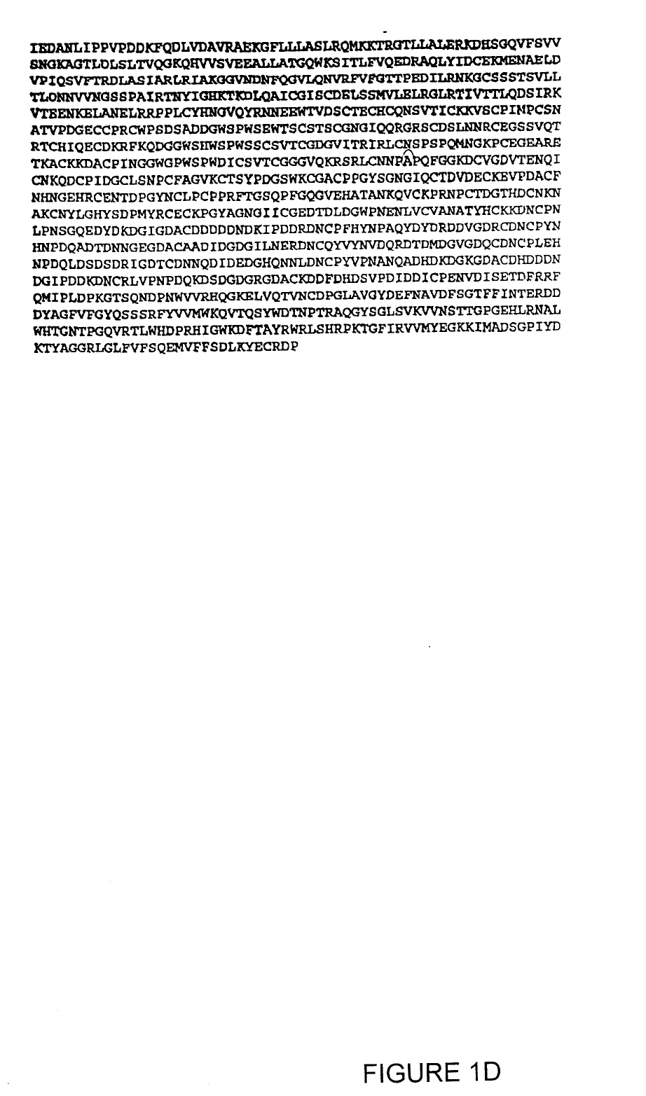

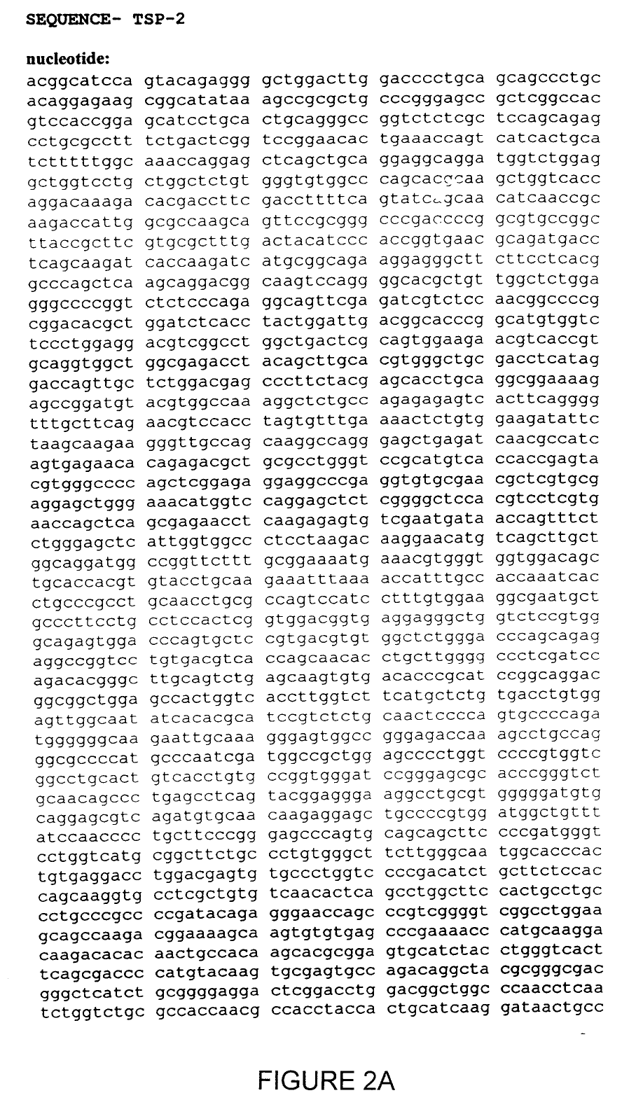

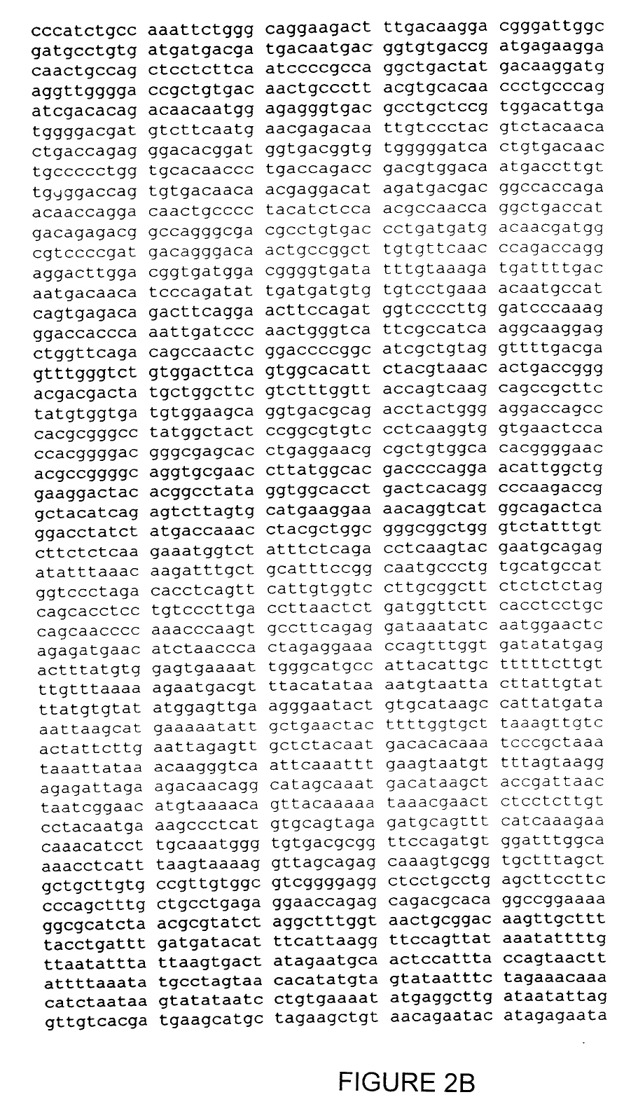

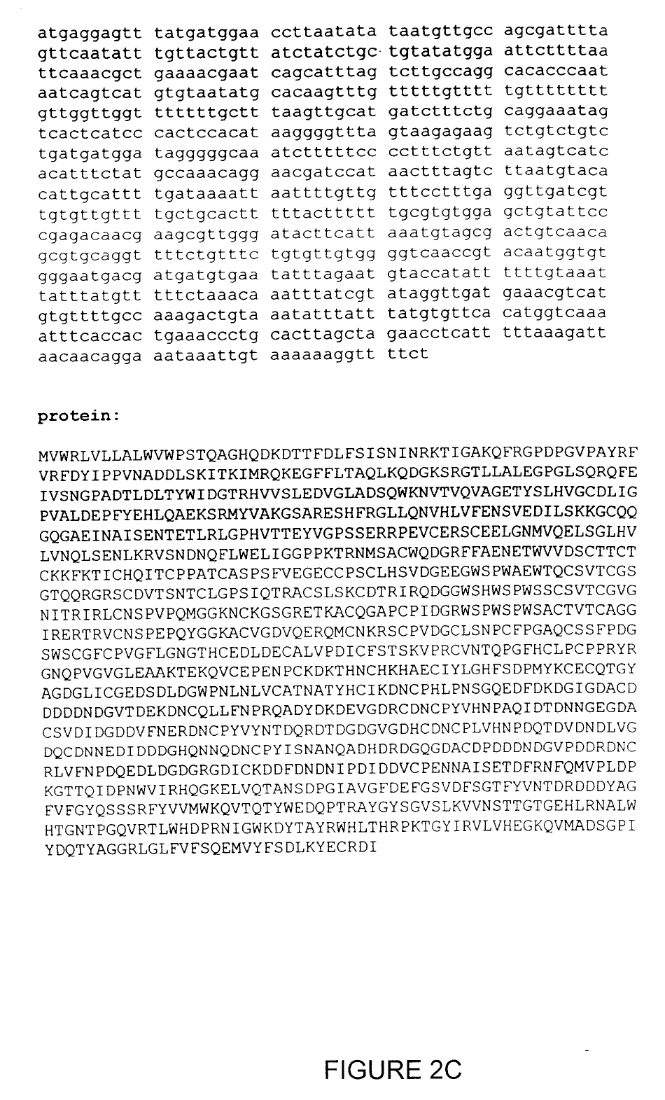

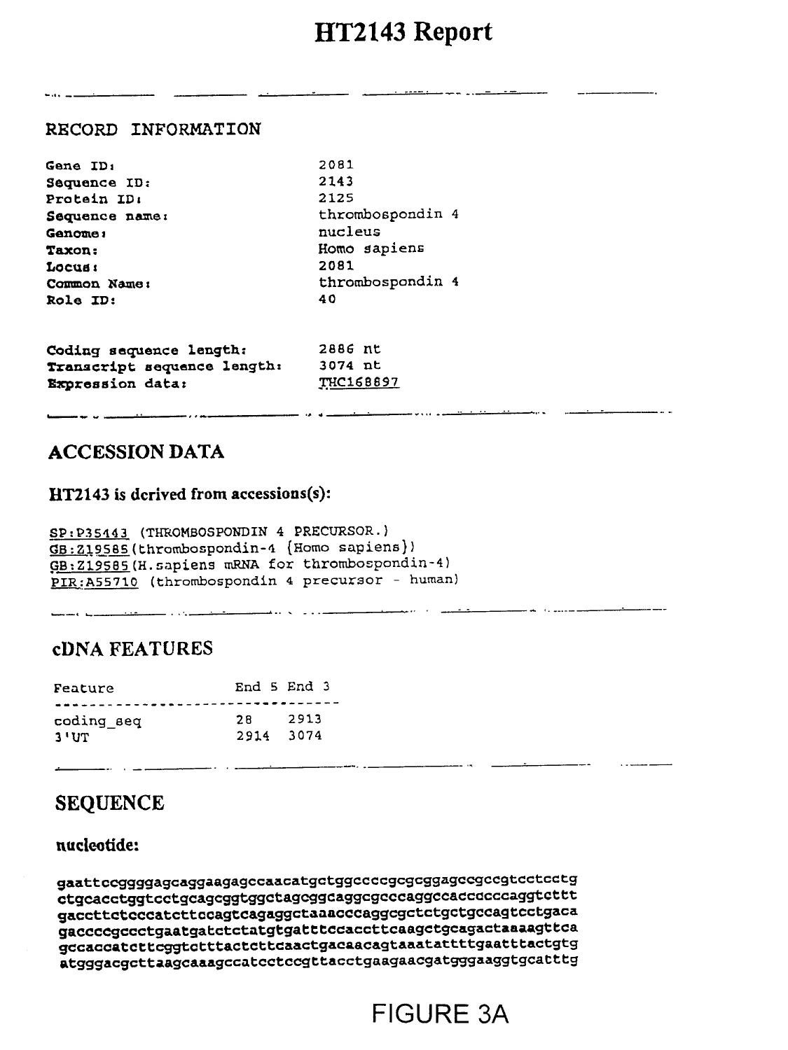

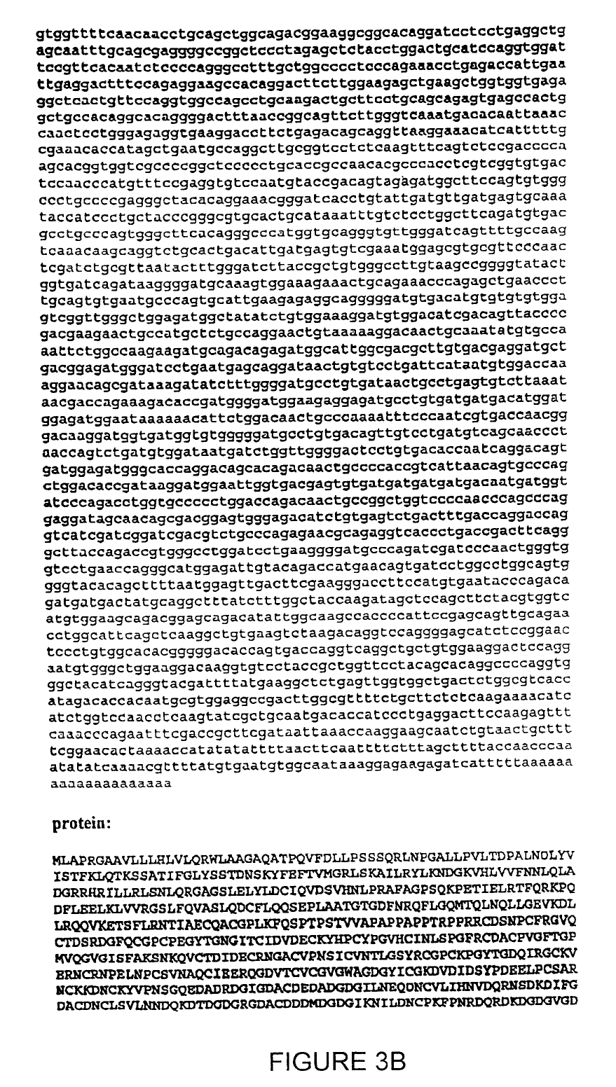

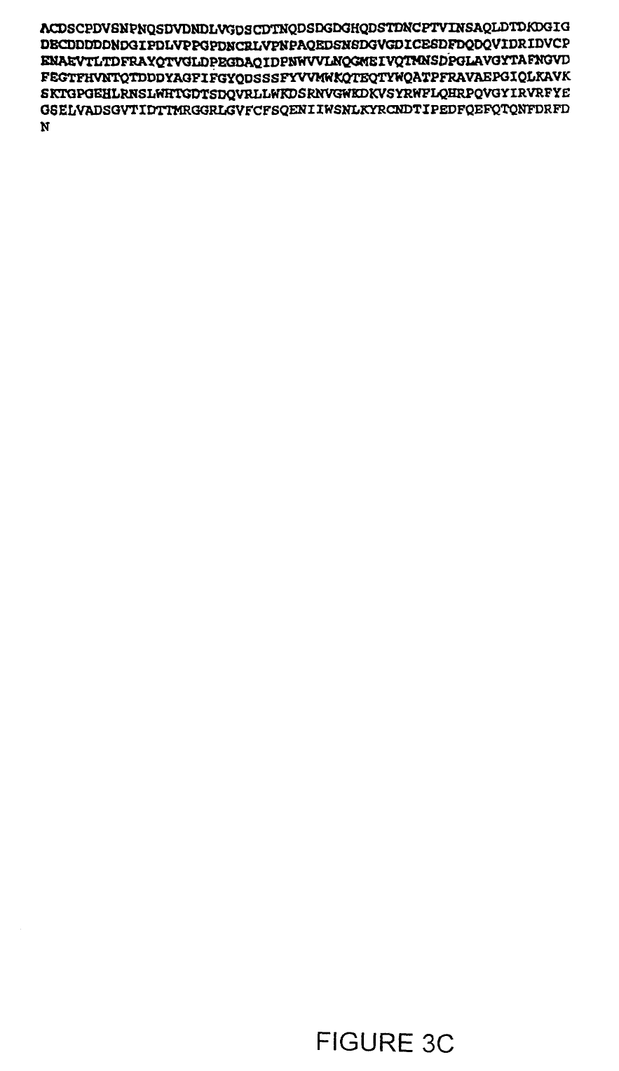

Single nucleotide polymorphisms (SNPs) were identified in TSP-1, TSP-2, and TSP-4 which are associated with premature cardiovascular disease, and, in particular, coronary artery disease (CAD) and myocardial infarction (MI). Specific reference nucleotide (SEQ ID NO: 1) and amino acid (SEQ ID NO: 2) sequences for TSP-1 are shown in FIGS. 1A-1D. Specific reference nucleotide (SEQ ID NO:5) and amino acid (SEQ ID NO:6) sequences for TSP-2 are shown in FIGS. 2A-2C. Specific reference nucleotide (SEQ ID NO: 3) and amino acid (SEQ ID NO: 4) sequences for TSP-4 are shown in FIGS. 3A-2C.

The first SNP (G334u4) is a change from A (adenine) (reference nucleotide) to G (guanine) (alternate or variant nucleotide) at nucleotide position 2210 of the nucleic acid sequence of TSP-1 (FIGS. 1A-1D), resulting in a missense amino acid mutation from asparagine (reference) to serine (alternate) at amino acid 700. The second SNP (G755e5) is a change from T (thymidine) (reference) to G (guanine) (alternate) at nucleotide position 3949 of the nucleic acid sequence of TSP-2 (FIGS. 2A-2C). This SNP is located in the 3′ untranslated region (UTR), near a highly conserved region which has a potential regulatory role (LaBell, et al. (1993) Genomics 17:225-229). The third SNP (G355u2) is a change from G (guanine) (reference) to C (cytidine) (alternate) at nucleotide position 1186 of the nucleic acid sequence of TSP-4 (FIGS. 3A-2C), resulting in a missense amino acid alteration from alanine (reference) to proline (alternate) at amino acid 387.

With respect to the G355u2 SNP, individuals with CAD carried at least one copy of the variant “C” allele more frequently than control individuals (43% as compared with 34%). With respect to the G355u2 SNP, individuals with MI carried at least one copy of the variant “C” allele more frequently than control individuals (49% as compared with 34%). With respect to the G5755e5 SNP, individuals with CAD carried one copy of the variant “G” allele more frequently than control individuals (46% vs. 39%) and individuals with MI carried one copy of the variant “G” allele more frequently than control individuals (49% vs 39%). With respect to the G334u4 SNP, individuals with CAD carried two copies of the variant “G” allele more frequently than control individuals (1.7% as compared with 0.2%). With respect to the G334u4 SNP, individuals with MI carried two copies of the variant “G” allele more frequently than control individuals (2% as compared with 0.2%).

### Example 2

Assessment of Thrombospondin Protein Levels

This example describes the measurement of the amount of thrombospondin protein present in a plasma sample by ELISA.

Plasma samples were obtained from 240 patients with MI or coronary revascularization before age 45 (men) or 50 (women). Cases were drawn (one per family) from a retrospective collection of sibling pairs with premature CAD. Cases were Caucasian. A complete database of phenotypic and laboratory variables for the affected patients afforded logistic regression to control for age, diabetes, body mass index, gender.

22 μl of 0.2 μg/μl P12 antibody was diluted in 11 ml of coating buffer (0.05 M carbonate buffer, pH 9.6), and mixed by pipetting. 100 μl of the diluted antibody was added to each well in a 96-well ELISA plate (Maxisorp Immunoplates, Nunc), covered, and incubated in the refrigerator (4° C.) for over night.

Each well was aspirated from the side and washed 3 times with 200 μl/well of 1× PBS. 200 μl per well of blocking buffer (1× PBS containing 0.1% Tween20 and 10 mg/ml of bovine γ-globulin) was added. The plate was covered and incubated at 37° C. (in a tissue culture incubator) for 90 min. The blocking buffer was quickly discarded. The plate was washed 3 times with 200 μl/well of 1× PBSTw (1× PBS containing 0.1% Tween20) and all liquids were completely drained.

Note: While blocking, standards and samples were prepared and stored on ice. Antigen buffer was used for diluting standards and samples (1× PBS containing 0.1% Tween20 and 10 mg/ml of bovine γ-globulin and 0.01 M EDTA). Only polypropylene tubes were used.

Sample preparation:

The plasma samples were thawed in a water bath at desired temperature (37° C. ) and mixed by gently inverting the vial five times. 150 μl of plasma was aliquoted into a polypropylene tube. 150 μl of antigen buffer was added and mixed well, but gently. The plasma was kept on ice until and during preparation of the samples and standards.

Standards:

A 15-μl vial of thrombospondin (200 ng/microliter) was thawed ice and spun briefly for 30 seconds in a microfuge. 500 μl of antigen buffer was added, and mixed. The solution was transferred carefully and quantitatively into a tube labeled as 2000 ng/ml. The vial was rinsed 2 more times with 500 μl of antigen buffer, each time transferring the wash to tube labeled as 2000 ng/ml. This was called “Standard Stock”. Thus, the final volume was 1500 μl. Using this as a guideline, calculation can be made for any number of plates.

Standard in Antigen Buffer:

1300 μl of “Standard Stock” was aliquoted into a new tube. 1300 μl of antigen buffer was added and mixed well for a concentration of 1000 ng/ml. 650 μl each was aliquoted into tubes labeled as “Standard 1, 2, 3 and 4”, referring to ELISA plates 1, 2, 3, and 4. 300 μl each was serially diluted with antigen buffer to concentration ranging from 1000, 500, 250, 125, 62.5, 31.2 and 15.6 ng/ml. They were plated in duplicate as per the template layout.

100 μl/well of standard or sample was add as per the template layout. The plate was covered and incubated at 37° C. (in a tissue culture incubator) for 60 min. The contents were discarded. The plate was washed 3 times with 200 μl/well of 1× PBSTw (1× PBS containing 0.1% Tween20). All liquids were drained completely. 5 μl of biotinylated P10 antibody was diluted in 12 ml of 0.2 μg/μl P12 antibody in 11 ml of blocking buffer (1× PBS containing 0.1% Tween20 and 10 mg/ml of bovine γ-globulin). 100 μl/well was added. The plate was covered and incubated at 37° C. (in a tissue culture incubator) for 60 min. The contents were discarded. The plate was washed 3 times with 200 μl/well of 1× PBSTw (1× PBS containing 0.1% Tween20). All liquids were drained completely. 24 μl of Streptavidin HRP was diluted in 12 ml of 1× PBSTw. 100 μl/well was added. The plate was covered and incubated at 37° C. (in a tissue culture incubator) for 45 min. During the last 15 min of incubation, about 12 ml of ABTS solution per plate were aliquoted, and it was brought to room temperature in dark.

The contents were discarded. The plate was washed 3 times with 200 μl/well of 1× PBSTw (1× PBS containing 0.1% Tween20). The plate was washed 3 times with 200 μl/well of 1× PBS. All liquids were drained completely. 100 μl/well of ABTS solution was added and incubated in dark for 15 min. 50 μl/well of 1.5% SDS was added to stop the reaction and the plate was mixed on a plate shaker for 2 min. The plate was read at 410 nm with a reference at 490 nm with Log-Lin sigmoid regression.

Note: Substrate solution ABTS is light and heat sensitive. Aliquot 12 ml/plate and leave at RT in dark for 15-20 min prior to use. Incubation may also be done in the dark for 15 min. Add 50 μl of 1.5% SDS to stop reaction. Mix on plate shaker for 2 minutes before reading.

240 patient plasma samples (previously collected in citrate tubes and stored at −70° C.) from the Gene Quest population were evaluated. All samples were analyzed in duplicate.

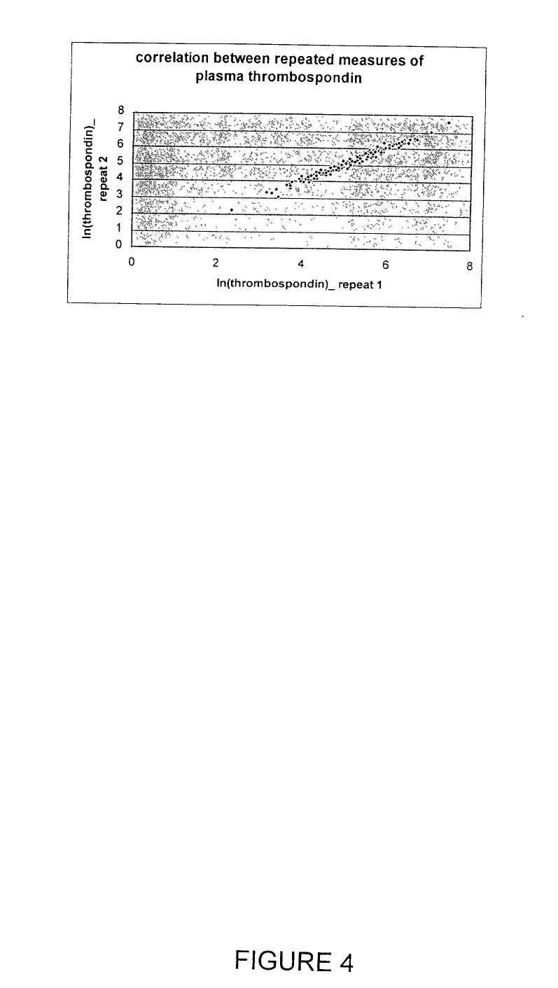

Repeat measures of plasma thrombospondin were strongly correlated over all ranges of thrombospondin (r²=0.96) (see FIG. 4).

Plasma thrombospondin levels ranged from 10-2031 and were not normally distributed and therefore needed to be log (ln)-transformed prior to statistical analysis.

### Example 3

Correlation of Plasma Thrombospond in Levels with Genotype

The association of genotypes at TSP-1, TSP-2 and TSP-4 with plasma levels of thrombospondin was performed using the GENMOD procedure in SAS statistical software. GENMOD fits a generalized linear model, taking into account repeated measures. In this case, the two repeated measurements of thrombospondin for each patient were taken into account. A model was fit where log-thrombospondin was the dependent variable and genotypes for all three genes entered into the model simultaneously.

TABLE 1
Results from multivariate analyses showing genotypes for the
thrombospondin genes, their association with myocardial
infarction, and correlation with level of plasma
thrombospondin in the GeneQuest population.

| | |
|---|---|
| Mean Plasma | P value |

| | | | |
|---|---|---|---|
| GENOTYPE | N | OR (MI) | TSP (ng/ml) |

| | | | | |
|---|---|---|---|---|
| THBS1_NN | 177 | 1.00 | 176 | — |
| THBS1_NS | 52 | 1.07 | 224 | .01 |
| THBS1_SS | 5 | 8.44 | 125 | .43 |
| THBS2_tt | 103 | | 179 | — |
| THBS2_tg | 106 | | 191 | .70 |
| THBS2_gg | 9 | | 215 | .63 |
| THBS4_AA | 133 | 1.00 | 200 | — |
| THBS4_AP | 94 | 1.79 | 164 | .13 |
| THBS4_PP | 14 | 1.54 | 187 | .85 |

N = number of patients
OR = odds ratio
MI = myocardial infarction
MEAN PLASMA = mean plasma level of thrombospondin
LN MEAN = natural log mean plasma (used in statistical analysis)

Based on these results, plasma levels of thrombospondin are significantly correlated with genotype at TSP-1 and suggestive of a correlation with TSP-4. The lack of association with TSP-2 does not indicate a lack of correlation between genotype and plasma level of thrombospondin, but rather may be due to the specificity of the antibodies used in this experiment. Because genetic variants in TSP-2 correlate with MI, it is believed that an assay that specifically detects the product of the TSP-2 gene (e.g., performing the assay of Example 2 with an antibody specific for TSP-2 protein) may also be used to demonstrate correlation between TSP-2 genotype and plasma levels of TSP-2 protein as well as correlation between TSP-2 protein level and risk of cardiovascular disease.

Furthermore, for both TSP-1 and TSP-4, the genotypes associated with the highest risk of MI also have the lowest levels of thrombospondin. Therefore, low levels of TSP may be correlated with increased risk of MI.

Other Embodiments

Those skilled in the art will recognize, or be able to ascertain using no more than routine experimentation, many equivalents to the specific embodiments of the invention described herein. Such equivalents are intended to be encompassed by the following claims.

What is claimed is:

1. A method of diagnosing or aiding in the diagnosis of a cardiovascular disease in a patient, the method comprising comparing:
a) the level of a thrombospondin marker in a sample, and
b) the normal level of thrombospondin marker in a control non-cardiovascular disease sample,
wherein a significant difference between the level of thrombospondin marker in the patient sample and the normal thrombospondin marker level is an indication that the patient is afflicted with or predisposed to cardiovascular disease.

2. The method of claim 1, wherein the sample is a blood fluid sample.

3. The method of claim 2, wherein the sample is plasma.

4. The method of claim 1, wherein the sample is obtained from a human subject.

5. The method of claim 1, wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

6. The method of claim 1, wherein the thrombospondin marker is selected from the group consisting of a TSP-1 nucleic acid molecule, a TSP-2 nucleic acid molecule, a TSP-4 nucleic acid molecule, and combinations thereof.

7. The method of claim 5, wherein the presence of the marker is detected using a reagent which specifically binds with a thrombospondin protein or fragment thereof.

8. The method of claim 7, wherein the reagent is selected from the group consisting of an antibody, an antibody derivative, and an antibody fragment.

9. A method for predicting the likelihood that an individual will have a cardiovascular disease, the method comprising comparing:
a) the level of a thrombospondin marker in a sample, and
b) the normal level of a thrombospondin marker in a control non-cardiovascular disease sample,
wherein a significantly different level of the thrombospondin marker in the sample, relative to the normal level, is an indication that the patient has an increased likelihood of having a cardiovascular disease.

10. The method of claim 9, wherein the sample is a blood fluid sample.

11. The method of claim 10, wherein the sample is plasma.

12. The method of claim 9, wherein the sample is obtained from a human subject.

13. The method of claim 9, wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

14. The method of claim 9, wherein the thrombospondin marker is selected from the group consisting of a TSP-1 nucleic acid molecule, a TSP-2 nucleic acid molecule, a TSP-4 nucleic acid molecule, and combinations thereof.

15. The method of claim 14, wherein the presence of the marker is detected using a reagent which specifically binds with a thrombospondin protein or fragment thereof.

16. The method of claim 15, wherein the reagent is selected from the group consisting of an antibody, an antibody derivative, and an antibody fragment.

17. A method for monitoring the progression of cardiovascular disease in a patient, the method comprising:
a) detecting in a patient sample at a first point in time, the level of a thrombospondin marker;
b) repeating step a) at a subsequent point in time; and
c) comparing the level of thrombospondin marker detected in steps a) and b), and therefrom monitoring the progression of cardiovascular disease in the patient.

18. The method of claim 17, wherein between the first point in time and the subsequent point in time the patient has undergone treatment for cardiovascular disease.

19. The method of claim 18, wherein the treatment is anticoagulant therapy.

20. The method of claim 17, wherein the sample is a blood fluid sample.

21. The method of claim 20, wherein the sample is plasma.

22. The method of claim 17, wherein the sample is obtained from a human subject.

23. The method of claim 17, wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

24. The method of claim 17, wherein the thrombospondin marker is selected from the group consisting of a TSP-1 nucleic acid molecule, a TSP-2 nucleic acid molecule, a TSP-4 nucleic acid molecule, and combinations thereof.

25. The method of claim 23, wherein the presence of the marker is detected using a reagent which specifically binds with a thrombospondin protein or fragment thereof.

26. The method of claim 25, wherein the reagent is selected from the group consisting of an antibody, an antibody derivative, and an antibody fragment.

27. A method of assessing the efficacy of a compound for inhibiting cardiovascular disease in a patient, the method comprising comparing:
a) the level of a thrombospondin marker in a first sample obtained from the patient and maintained in the presence of the compound, and
b) the level of a thrombospondin marker in a second sample obtained from the patient and maintained in the absence of the compound,
wherein a significantly enhanced level of a thrombospondin marker in the first sample, relative to the second sample, is an indication that the compound is efficacious for inhibiting cardiovascular disease in the patient.

28. The method of claim 27, wherein the sample is a blood fluid sample.

29. The method of claim 28, wherein the sample is plasma.

30. The method of claim 27, wherein the sample is obtained from a human subject.

31. The method of claim 27, wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

32. The method of claim 27, wherein the thrombospondin marker is selected from the group consisting of a TSP-1 nucleic acid molecule, a TSP-2 nucleic acid molecule, a TSP-4 nucleic acid molecule, and combinations thereof.

33. The method of claim 31, wherein the presence of the marker is detected using a reagent which specifically binds with a thrombospondin protein or fragment thereof.

34. The method of claim 33, wherein the reagent is selected from the group consisting of an antibody, an antibody derivative, and an antibody fragment.

35. The method of claim 27, wherein the first and second samples are portions of a single sample obtained from the subject.

36. The method of claim 27, wherein the first and second samples are portions of pooled samples obtained from the subject.

37. A method of assessing the efficacy of a therapy for inhibiting cardiovascular disease in a patient, the method comprising comparing:
a) the level of a thrombospondin marker in the first sample obtained from the patient prior to providing at least a portion of the therapy to the patient, and
b) the level of a thrombospondin marker in a second sample obtained from the patient following provision of the portion of the therapy,
wherein a significantly enhanced the level of a thrombospondin marker in the second sample, relative to the first sample, is an indication that the therapy is efficacious for inhibiting cardiovascular disease in the patient.

38. The method of claim 37, wherein the therapy is anticoagulant therapy.

39. The method of claim 37, wherein the sample is a blood fluid sample.

40. The method of claim 39, wherein the sample is plasma.

41. The method of claim 37, wherein the sample is obtained from a human subject.

42. The method of claim 37, wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

43. The method of claim 37, wherein the thrombospondin marker is selected from the group consisting of a TSP-1 nucleic acid molecule, a TSP-2 nucleic acid molecule, a TSP-4 nucleic acid molecule, and combinations thereof.

44. The method of claim 42, wherein the presence of the marker is detected using a reagent which specifically binds with a thrombospondin protein or fragments thereof.

45. The method of claim 44, wherein the reagent is selected from the group consisting of an antibody, an antibody derivative, and an antibody fragment.

46. A kit for assessing whether a patient is afflicted with or predisposed to cardiovascular disease, the kit comprising reagents for assessing the level of a thrombospondin marker.

47. The kit of claim 46 wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

48. The kit of claim 46 wherein the thrombospondin marker is selected from the group consisting of a TSP-1 nucleic acid molecule, a TSP-2 nucleic acid molecule, a TSP-4 nucleic acid molecule, and combinations thereof.

49. A kit for assessing the suitability of a compound for inhibiting cardiovascular disease in a patient, the kit comprising:
a) the compound; and
b) a reagent for assessing expression of a thrombospondin marker.

50. The kit of claim 49, wherein the thrombospondin marker is selected from the group consisting of a TSP-1 protein, a TSP-2 protein, a TSP-4 protein, a fragment of a TSP-1 protein, a fragment of a TSP-2 protein, a fragment of a TSP-4 protein, and combinations thereof.

51. The kit of claim 49, wherein the thrombospondin marker is selected from the group consisting of a TSP-1 nucleic acid molecule, a TSP-2 nucleic acid molecule, a TSP-4 nucleic acid molecule, and combinations thereof.
